(19) United States
(12) Patent Application Publication
(10) Pub. No.: US 2023/0092685 A1
(43) Pub. Date: Mar. 23, 2023

(54) SYSTEMS AND METHODS FOR SECURELY MANAGING PERSONAL INFORMATION USING TRUSTED LEDGERS

(71) Applicant: Intertrust Technologies Corporation, Milpitas, CA (US)

(72) Inventors: David Maher, Philadelphia, PA (US); Julian Durand, Ladysmith (CA); Michael Manente, Sudbury, MA (US); Eric Swenson, Soquel, CA (US)

(73) Assignee: Intertrust Technologies Corporation, Milpitas, CA (US)

(21) Appl. No.: 17/948,827

(22) Filed: Sep. 20, 2022

Related U.S. Application Data
(60) Provisional application No. 63/261,415, filed on Sep. 20, 2021.

(51) Int. Cl. G06F 21/62; G16H 10/60; G06F 21/60

(52) U.S. Cl. CPC G06F 21/6245; G16H 10/60; G06F 21/602

(57) ABSTRACT

The disclosure relates to, among other things, systems and methods for securely managing personal information, which may include vaccination and/or other health status information. Aspects of the disclosed embodiments may use trusted ledgers and/or ledger derivatives in connection with managing personal information. Various mechanisms are described which facilitate cooperation and trust between various certification authorities, which may comprise public health authorities, allowing for cross jurisdictional verification of certified personal information.

### RELATED APPLICATIONS

This application claims the benefit of priority under 35 U.S.C. § 119(e) to U.S. Provisional Patent Application No. 63/261,415 filed Sep. 20, 2021, and entitled “SYSTEMS AND METHODS FOR SECURELY MANAGING PERSONAL INFORMATION USING TRUSTED LEDGERS,” the contents of which is hereby incorporated by referenced in its entirety.

### COPYRIGHT AUTHORIZATION

Portions of the disclosure of this patent document may contain material which is subject to copyright protection. The copyright owner has no objection to the facsimile reproduction by anyone of the patent document or the patent disclosure, as it appears in the U.S. Patent and Trademark Office patent file or records, but otherwise reserves all copyright rights whatsoever.

### SUMMARY

The present disclosure relates generally to systems and methods for securely managing personal information. More specifically, but not exclusively, the present disclosure relates to systems and methods for managing personal information, which may include vaccination and/or other health status information, using trusted ledgers and/or ledger derivatives.

In what may be remembered as one of humanity's most phenomenal accomplishments, vaccines for the SARS-CoV-2 virus have been developed and delivered in record time. Due to logistical challenges, delivery of these vaccines has been non-uniform, which coupled with vaccine hesitancy amongst certain constituencies means that the world may have to live with the reality of a partially immune population for years to come. Using conventional mechanisms, it may be difficult to reliably determine if an individual is vaccinated or not. As such, relaxing restrictions on public gatherings and/or travel presents challenges. Indeed, the population of the world is simply too big, too distributed, and has too wide-ranging vaccine uptake acceptance tolerance for simultaneous vaccination of the entire world community.

Certain non-pharmaceutical interventions (“NPIs”) for managing pandemic disease, including what may be described generally as “lockdowns” or other associated business and/or gather restrictions, have certain costs. Humans crave communal gatherings and businesses need their customers to survive long term. Families desire to travel between borders to see loved ones, and international business travel and diplomacy depend in part on the relatively free flow of people and/or goods across borders.

Credentials that establish vaccination status, which may be generally known as “vaccination passports,” “vaccination cards,” and/or derivatives of the same, have been considered as a means of managing the process of opening society in partially vaccinated communities, and have historic precedence. For example, the United Nations played a major role in providing credentials that simultaneously helped increase inoculations against polio and smallpox and ease global travel from the 1950s onwards. As the world became healthier and infectious diseases became more of a nuisance than an exigent concern, many nations eased their requirements on the paper documents that travelers needed to cross borders. A few nations replaced these requirements with public health checkpoints where travelers passed through a screening process that often-used thermal imaging to identify symptomatic disease.

The Covid-19 crisis has brought back the question of whether to use vaccination passports again. Ironically, in a world never more globalized entering a transnational era where people around the planet bond over social networks, the urge to re-engage physically, which can be more safely enabled by vaccination credentials, is being throttled by privacy concerns, national data control, and/or residency issues.

Unlike in the 1950s, intranational and international consensus is harder to achieve, despite the deployments of a global, open, and interoperable communication network and a massive global population accustomed to interconnection.

Embodiments of the disclosed systems and methods may be used to securely manage personal information, including vaccination status information, in a manner that allows for NPIs for pandemic control to be applied in a more targeted fashion without compromising individual privacy. In addition, aspects of the disclosed embodiments may allow for cross-jurisdictional verification of certified personal information. In connection with various embodiments of the systems and methods disclosed herein, individuals may maintain both their privacy and freedom to congregate at the same time, while preserving public health and general well-being. Moreover, health authorities (e.g., the U.S. Center for Disease Control (“CDC”), the World Health Organization (“WHO”), and/or other health ministries) may be better able to coordinate implementation of NPIs via cross-jurisdictional platforms.

Various embodiments of the disclosed systems and methods may use trusted ledgers. Trusted ledgers, which may comprise public trusted ledgers, may be used to securely record and/or manage a variety of information. For example, secure ledgers may record and/or manage information that may be used to determine information provenance, provide anonymous proof of attribution, ownership, and/or creation, provide the public a trusted way to audit and/or otherwise verify certain claims and/or assertions, and/or the like.

Consistent with various embodiments disclosed herein, trusted databases, ledgers, and/or the like, may be used to record and/or otherwise manage various assertions, bindings, attributes, identities, and/or the like associated with individuals (e.g., vaccination status information) and/or various entities. In some embodiments, trusted ledgers may be distributed in nature. Distributed trusted ledgers may be referred to in certain instances herein as trusted immutable distributed assertion ledgers (“TIDALs”) and/or variations of the same. Ledgers may, in various embodiments, be public, private, and/or a combination thereof. In certain embodiments, a TIDAL may comprise a public indelible distributed database (“PIDD”). TIDALs may be associated with a variety of properties including, for example, ledger processes that may be resistant to byzantine failures, entries that may be immutable and/or relatively immutable, entries that may be time-synced (at least in part), entries that may be scalable, and/or entries that may be available for relatively fast lookup.

Trusted ledgers, including TIDALs, may be implemented, at least in part, using various blockchain technologies. Users of trusted ledgers may post entries into a sequenced database. Each entry may have an associated message, a digital signature of the message (which may be referred to herein in certain instances as simply a “signature” and/or derivatives of the same), and some associated verification key which others can use to verify that a person with the verification key's associated signing key has signed the message.

In various embodiments of the disclosed trusted ledgers, entries may be appended to the ledger. Each addition of an entry may be witnessed by a number of parties, which may be referred to in certain instances herein as ledger nodes, and entries may be accompanied by various auxiliary cryptographic information to ensure that changes to messages in the database and/or the ordering of entries can be detected. Entries may reside in a unique numerical position in the ledger, and once all witnesses agree to add an entry to the ledger, it is difficult to alter an entry's presence, contents, and/or position in the ledger without detection by the witnesses.

Witnesses may take a variety of forms. For example, in blockchain-based cryptographic currencies, a witness may be any suitably configured computer. In other implementations, permissioned systems associated with independent entities and/or companies joined in a consortium may operate as witnesses.

Although various embodiments and/or examples described herein relate to managing information relating to vaccination status of individuals, it will be appreciated that embodiments of the disclosed systems and methods are not so limited. Indeed, embodiments disclosed herein may be used in connection with a variety of types of personal information and/or other contexts, applications, and/or use cases.

### BRIEF DESCRIPTION OF THE DRAWINGS

The inventive body of work will be readily understood by referring to the following detailed description in conjunction with the accompanying drawings, in which:

FIG. 1 illustrates a non-limiting example of the management of a trusted ledger consistent with certain embodiments disclosed herein.

FIG. 2 illustrates a non-limiting example of an architecture for recording and validating vaccination status of an individual using a plurality of ledgers consistent with certain embodiments disclosed herein.

FIG. 3 illustrates another non-limiting example of an architecture for authenticating a gatekeeping application using a plurality of ledgers consistent with certain embodiments disclosed herein.

FIG. 4 illustrates a flow chart of a non-limiting example of a method for verifying personal health status information associated with a user consistent with certain embodiments of the present disclosure.

FIG. 5 illustrates a flow chart of a non-limiting example of a method for authenticating a user application consistent with certain embodiments of the present disclosure.

FIG. 6 illustrates a non-limiting example of a system that may be used to implement certain embodiments of the systems and methods of the present disclosure.

### DETAILED DESCRIPTION

A detailed description of the systems and methods consistent with embodiments of the present disclosure is provided below. While several embodiments are described, it should be understood that the disclosure is not limited to any one embodiment, but instead encompasses numerous alternatives, modifications, and equivalents. In addition, while numerous specific details are set forth in the following description in order to provide a thorough understanding of the embodiments disclosed herein, some embodiments can be practiced without some or all of these details. Moreover, for the purpose of clarity, certain technical material that is known in the related art has not been described in detail in order to avoid unnecessarily obscuring the disclosure.

The embodiments of the disclosure may be understood by reference to the drawings. The components of the disclosed embodiments, as generally described and illustrated in the figures herein, could be arranged and designed in a wide variety of different configurations. Thus, the following detailed description of the embodiments of the systems and methods of the disclosure is not intended to limit the scope of the disclosure, as claimed, but is merely representative of possible embodiments of the disclosure. In addition, the steps of any method disclosed herein do not necessarily need to be executed in any specific order, or even sequentially, nor need the steps be executed only once, unless otherwise specified.

Embodiments of the disclosed systems and methods may be used to securely manage personal information, which may comprise personal health information such as, for example and without limitation, vaccine status information. In some embodiments, vaccination status information may be managed in a manner that allows for NPIs for pandemic control to be applied in a more targeted fashion without compromising individual privacy. Aspects of the disclosed embodiments may allow for cross-jurisdictional verification of personal information certification. Various aspects of the disclosed systems and methods may address personal information management and privacy challenges using, at least in part, trusted ledgers and/or associated derivative ledgers.

### Vaccination Information Management Challenges

Across the globe, nations, their local governments, and corporations are attempting to introduce their own vaccine passports, striving to balance personal privacy with public and/or workforce health concerns. With different formats, issued from authorities in different legal regimes, it may be difficult for cross entity and/or jurisdictional verification and/or certification of vaccine passports. For example, with a traditional vaccine passport, it may be difficult for a Japanese vaccine passport to be accepted by an American gatekeeper at a sports stadium venue. Embodiments of the disclosed systems and methods may allow for trusted cross entity and/or cross jurisdictional verification and/or validation of passports and/or certificates relating to personal information, such as vaccination status.

In various embodiments, mechanisms are described that facilitate secure management of personal information while protecting individual privacy. In some embodiments of the disclosed systems and methods, concerns relating to undesired tracking and differential trust between entities (e.g., between a national/domestic governmental entity and a foreign governmental entity) may be addressed. Further aspects of the disclosed embodiments may mitigate forgery of certificates attesting to certain personal information such as vaccination status. For example, a certificate from a legitimate authority may have its data tampered with, a legitimate authority may be spoofed (or both may occur), and/or the like. Paper and/or other conventional certificates may be copied and/or modified with relative ease without significant tamper proofing features, and thus may be susceptible to forgeries.

Consistent with embodiments disclosed herein, various trusted ledger technologies, which in some implementations may use blockchain ledgers, may be used to securely associate an individual's identity to certain personal information such as vaccination status. Many conventional technologies, however, are relatively inefficient and are difficult to scale to support millions of concurrent transactions. For example, proof-of-work and proof-of-stake consensus protocols used in blockchains supporting many crypto-currencies are computationally intensive, costly, and require large amounts of energy consumption to support. Moreover, an entity controlling a majority of stakeholders may reduce trust in the ledger given the potential for single entity influence. Blockchain technologies using a single authority may not be well suited for interoperability between other blockchain systems and/or may be associated with a single point of failure.

Embodiments of the disclosed systems and methods provide for a more responsive, scalable, less-hierarchical, distributed trust management fabric comprising of heterogeneous deployments of lightweight, distributed trusted ledgers that in some implementations may be independently operated. In certain embodiments, each ledger may be dedicated to supporting specific kinds of trust assertions, which may be also referred to in certain instances herein as bindings, from trusted knowledge sources and capable of rapidly responding to market needs.

In connection with various aspects of the disclosed systems and methods, trusted services may be used to verify assertions and/or statements offered for recordation in a trusted ledger—for example, the vaccination status of a particular individual. Embodiments of the disclosed systems and methods may help ensure one or more of:
The provenance, legitimacy, and/or authenticity of an authority making an assertion.
The definition of an assertion from an authority satisfies criteria of a trusted service validating the assertion.
The assertion is immutable and not easily forged.
Individuals may be identified uniquely without revealing personal details.

For example and without limitation, in context of a hypothetical Japanese individual named Sakura, embodiments of the disclosed systems and methods may use a trusted service to verify a statement from the Japanese government for recordation in a ledger that Sakura is fully vaccinated, that the Japanese government is a legitimate authority for making the assertion, and/or that the definition of “fully vaccinated” by the Japanese government meets the criteria of the trusted service.

### Trusted Assertion Ledgers

Trusted assertion ledgers consistent with embodiments disclosed herein may be lightweight, resilient, and easily distributed under governance of trusted services. In certain embodiments, trusted services may be generally referred to herein as trusted authorities. Trusted assertion ledgers may offer trusted data to interested parties without revealing more than what is needed by the interested parties in connection with an informational transaction. For example, in connection with the above hypothetical individual, the assertion that Sakura is properly vaccinated need not expose Sakura's identity and/or any other private information. A recorded trusted vaccination status assertion associated with Sakura, which may function as a digital vaccine certificate, may simply provide that its associated user and/or holder (whose identity may be anonymized) is vaccinated as asserted by a trust authority.

Assertions relating to personal information recorded in trusted ledgers consistent with various aspects of the disclosed embodiments may take a variety of forms and/or use a variety of suitable sematic structures. For example and without limitation, in various disclosed embodiments, an Assertion (A) about a subject made by Entity (E) may be hashed and signed by E with private key (S) and entered into a trusted ledger. In at least one non-limiting example in connection with the previously described hypothetical, an assertion may take the form:
Fact [A]=Sakura has been fully vaccinated per vaccine manufacturer guidelines.
Entity [E]=Japanese Ministry of Health.
Private Key [S]=Japanese Ministry of Health private key.
Ledger Entry and/or Assertion: Fact A made by entity E may be hashed and signed by E with private key S and entered into a trusted ledger. A ledger entry and/or an assertion may be generated in a variety of suitable ways consistent with various aspects of the embodiments disclosed herein. For example and without limitation, in some embodiments, an entry and/or assertion may comprise a hash of fact A and signature of fact A by entity E with private key S. In further embodiments, an entry and/or an assertion may comprise a hash of fact A and a signature of the hash of fact A by entity E with private key S. In yet further embodiments, an entry and/or an assertion may comprise fact A, a hash of fact A, and a signature by entity E with private key S of the hash of fact A. It will be appreciated that ledger entries and/or assertions may be generated in a variety of ways using a number of suitable semantic structures and include a variety of information, which may depend on a particular application and/or context, and that the examples presented herein are to be considered as illustrative and non-limiting.

Assertions in the trusted ledger may be verified, for example, by verifying the public key (P) of the Japanese government associated with the private key (S) is valid according to trusted authorities, which may include other Japanese Federal entities, as well as other validators such as the U.S. government. The root policy of the ledger may assert E is trusted to make assertions according to trusted authorities A1, A2 . . . A[n].

### Trusted Assertion Ledger Architecture and Management

Trusted ledgers consistent with various aspects of the disclosed embodiments, including TIDALs, may be associated with a variety of properties that may include one or more of:
Trusted—The ledger (and/or derivative ledgers) may be trusted due to integrity-assured and/or authenticity-assured assertions recorded in the ledger.
Immutable—What is recorded cannot be unrecorded, helping to provide Perfect Forward Integrity properties for assertions and/or bindings. This may help with auditing of trusted transactions.
Distributed—Data stores may be distributed and/or rely on intrinsic cryptographic mechanisms for their resiliency, rather than integrity being maintained solely by being stored in a protected enclave. Trust may be distributed and/or peer-to-peer as compared to traditional hierarchical PKIs.

Trusted ledgers consistent with various aspects of the disclosed embodiments, including TIDALs, may be associated with a variety of further properties that may include one or more of:
Ledger processes that may be resistant to byzantine failures.
Entries that may be time synchronized (at least in part). Distinguished sets of new entries (e.g., such as blocks in a blockchain) may have an immutable ordering whereby newer (e.g., more recent in actual time) entries may be relatively higher in order than earlier entries. In some instances, entries may be timestamped to identify a specific time of entry.
Ledgers may be scalable in number of entries.
Entries in a ledger may be available for relatively fast lookup and/or search.

In certain embodiments, scalability, and fast lookup and/or search may be achieved and/or otherwise improved by using derivatives of a ledger, if not by the ledger itself.

Consistent with various disclosed embodiments, trusted ledger paradigms may comprise permissioned blockchains that use efficient byzantine agreement protocols. In some embodiments, ledger entries may comprise assertions, made by a class of qualified submitters, that each binds a key (and/or a hash or other derivative of a key) with other attributes that are associated with that key, such as the identity of the owner and/or an alias thereof (e.g., an account), the scope of authority of the owner, information rights management permissions, and/or the like.

A ledger may be distributed among a plurality of nodes. A full node may have a full copy of the ledger. In certain embodiments, ledger actors, nodes, and/or entities may include assertion submitters, witnesses and/or verifiers, and/or distributed ledger node operators.

In certain implementations, a number of ledgers may be employed, each of which may specialize in the recording of various types of assertions with appropriate policies for the associated assertion types. In some embodiments, a given application may rely on the authenticity of multiple assertions and may either directly and/or indirectly query multiple ledgers. For example, a ledger may be indirectly queried when a ledger derivative is queried. In some embodiments, a ledger derivative may comprise one or more databases and/or ledgers derived from information recorded in one or more other ledgers.

In some embodiments, ledgers may be used to collect assertions and/or evidence of authority for a node that affirms such information, allowing multiple parties to cross check for compliance with policy. Ledgers may record the authentication information (e.g., a hash of the assertion) in public parts of the database and/or ledger. In certain instances, some applications may record the hash of encrypted information. Other applications, however, may record the hash of the information plaintext in a way such that access to the authentication information may be governed and/or may be modified (e.g., nullified). Access to unhashed plaintext information may be governed by applicable policies.

In some embodiments, a policy may be established that permits certain trusted authorities to update or revoke assertions stored in a trusted ledger. A policy may allow the original authority to perform such actions and/or may permit a delegate, a replacement, and/or a supervisor to do so. If permitted by policy, an updated assertion may be recorded by adding a new assertion to the trusted ledger, labeling it as an update and/or an outright revocation, potentially including a back pointer to the previous assertion. A ledger derivative then may (1) remove the hash of the original assertion from the derivative or (2) add a flag (to the original assertion or hash) together with a forward pointer from the revoked assertion to the new assertion stating it was revoked or updated, and/or a flag and backward pointer from the new assertion of revocation or update to the old assertion). In certain embodiments, the derivative may provide the latest truth about a putative assertion, when queried, but in some embodiments additional history may also be available for the response to a query.

In embodiments that permit assertions to be updated and/or revoked, the trusted entity performing the update and/or modification may receive an updated assertion as a digitally signed message and/or command from an entity with the (purported) authority to request the update. The digitally signed message and/or command may include a hash for verifying the integrity of the message content and a public key and/or certificate for verifying the authenticity of the signature. After verifying the message for authenticity, the trusted service may evaluate its policy and may query a trusted ledger to evaluate the authority of verified message/command issuer to update or modify an assertion. Provided that the validated issuer of the update and/or revocation message meets the requirements of the policy, the trusted entity may proceed to update and/or revoke the assertion using a process such as previously described.

In certain embodiments, a ledger may comprise a blockchain, although other database and/or ledger structures may be used. For example, hash graphs, tangles or directed, acyclic graphs and/or the like may also be used in connection with various aspects of the disclosed embodiments. In some embodiments, ledgers may be publicly readable, but in other embodiments they may not necessarily be publicly readable. For example, in connection with various aspects of the disclosed embodiments, ledgers may not necessarily be publicly accessible in every application, with some applications using multiple ledgers, some of which may be public and some private. Furthermore, in some embodiments, a ledger may be replaced and/or used in conjunction with a database that lacks some of the properties of a ledger, as may be the case for parts of a protocol where distributed trust is not necessarily required. In additional embodiments, information included in ledgers may be trusted through a variety of suitable cryptographic protection and/or other security techniques used in connection with recording, maintaining, and/or querying ledgers.

In some embodiments, trusted ledgers may be implemented using relatively flat data structures that may be open and/or may have portions protected for confidentiality depending on circumstances. Synchronized with other ledgers, a distributed ledger system may offer relatively high levels of availability and/or currency of data. In some embodiments, ledgers may be used to record assertions relating to the confidentiality of information included in the ledger. For example, in the illustrative case above, an assertion by the Electronic Frontier Foundation (“EFF”), the EU, and/or a federal and/or local authority may record an assertion in a ledger that the assertion relating to the vaccination status is deemed by that authority to be anonymous and complies with EFF policies, GDPR, CCPA, and/or the like. Consistent with embodiments disclosed herein, other assertions recorded in a trusted ledger may be referenced to validate a subject assertion.

### Trusted Ledger Derivatives

A trusted ledger derivative may comprise a trusted ledger that includes entries that are generated and/or otherwise derived using entries from another ledger. In various embodiments, a trusted ledger derivative may allow for relatively fast and/or otherwise efficient lookup of entries while also protecting sensitive and/or otherwise personal information. A trusted ledger derivative may comprise an index and/or a hash table (e.g., an ordered hash tables) allowing for easy reference to a subset of records. In some embodiments, trusted ledger derivatives may be distributed and/or synchronized, allowing for cross-checking with other ledgers and/or ledger derivatives. In further embodiments, trusted ledger derivatives may be generated and/or maintained by entities focusing on specific types of applications and/or use cases.

In various embodiments, hashes may be used to generate entries included in a derivative ledger. A hash function may comprise a cryptographic one-way function that outputs a fingerprint of the data that is provided to the function as an input. Thus, in the above non-limiting example, the presence of a hash of an assertion that Sakura is fully vaccinated may be looked up in a derivative ledger for verification, which may be further verified by accessing a trusted ledger the derivative ledger was derived from.

### Trusted Ledger Architecture

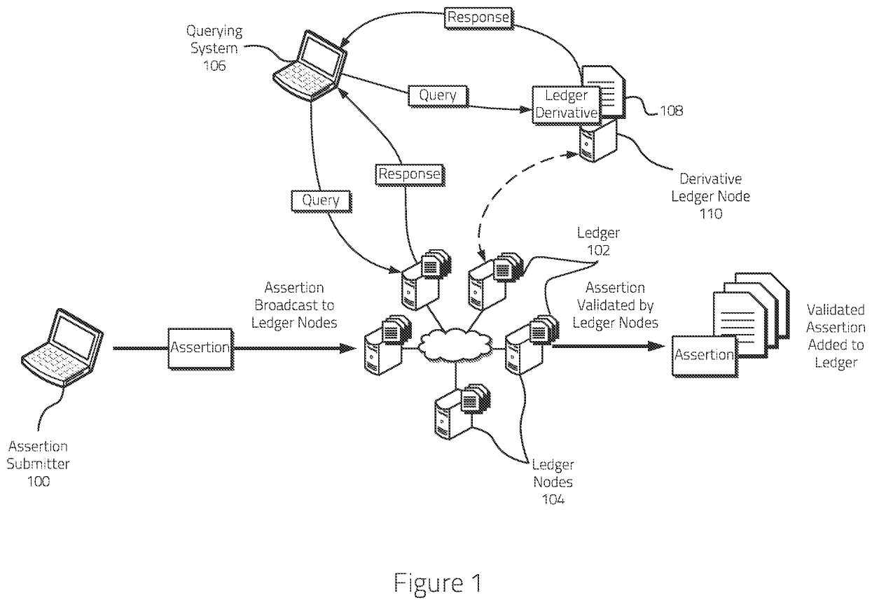

FIG. 1 illustrates an example of the management of a trusted ledger 102 consistent with certain embodiments disclosed herein. In certain embodiments, the trusted ledger 102 may be distributed in nature such as, for example and without limitation, in the case of a TIDAL, although aspects of the disclosed embodiments may also be used in connection with and/or any other suitable type of distributed database and/or ledger in any suitable form.

As illustrated, an assertion submitter 100 may submit an assertion for recordation in the ledger 102. In some embodiments, the assertion submitter 100 may comprise an entity and/or system that possesses credentials indicating authority to submit assertions for consideration to be recorded in the ledger 102. For example, in some embodiments, the assertion submitter 100 may comprise a user system, a name authority and/or other authority, and/or the like.

It will be appreciated that an assertion may comprise any type of data and/or information that may be recorded in a ledger, including any of the types of data and/or information described in connection with the various examples described herein. In some embodiments, an assertion may comprise associative information describing a relationship between different data and/or information. For example and without limitation, an assertion may comprise an association between data and/or information that identifies a user and/or an entity with fact information associated with the user and/or entity (e.g., vaccination status).

In some embodiments, an assertion may be generated based on a transformation of the subject associated data and/or information and/or portions thereof. For example and without limitation, an assertion may comprise a hash generated based on data and/or information that is the subject of the assertion. Other types of transformations are also contemplated. Thus it will be appreciated that various examples and embodiments of assertions described herein are provided for purposes of illustration and explanation, and not limitation.

In some embodiments, a submitted assertion may comprise an identifier associated with the assertion submitter 100 that may be used by various ledger nodes 104 in connection with a verification and/or witnessing process to determine whether the assertion submitter 100 has the requisite authority to make a submission of the specific type and/or with the scope reflected in the submitted assertion for inclusion in the ledger 102.

The submitted assertion may be broadcast to various ledger nodes 104 that may, among other things, maintain and/or otherwise manage the ledger 102 and in certain instances herein may be referred to as ledger management systems and/or nodes. In certain embodiments, at least a portion of ledger nodes 104 may be configured to verify submitted assertions prior to recordation of the assertions in the ledger 102. Consistent with various disclosed embodiments, assertions may be entered into the ledger 102 upon the agreement of multiple ledger nodes 104 operating as witnesses and/or verifiers. The ledger nodes 104 operating as witnesses and/or verifiers may verify the authenticity of the authority of the assertion submitter 100 to verify that the putative authority is in fact authorized to make the assertion in accordance with one or more applicable policies. In some embodiments, to verify the authenticity of the authority of the assertion submitter 100 to make the submitted assertion, the ledger nodes 104 may check previous entries in the ledger 102 and/or entries in other ledgers and/or associated ledger derivatives to verify that the assertion submitter 100 is authorized to make the submission to the ledger in accordance with applicable policy.

The ledger nodes 104, operating as witnesses, may verify a variety of information prior to recording a submitted assertion in the ledger 102. For example, the ledger nodes 104 may verify that an identifier submitted with the assertion (e.g., an ID of the assertion submitter 100, a public key, and/or the like) is valid and/or has not been revoked. The ledger nodes 104 may further verify that the submitter's scope of authority includes authority over the subject of the assertion. In certain embodiments, this may involve verifying prior submissions regarding the assertion submitter 100 included in the ledger 102.

In various embodiments, verified assertions may be placed into a pool to be entered into the ledger 102, and when a threshold number of ledger nodes 104 operating as witness agree regarding the authenticity and/or the actual authority of the assertion submitter 100 to make the assertion, the assertion may be recorded in the ledger 102. In certain embodiments, this agreement may be reached in accordance with an applicable agreement policy using, for example and without limitation, a byzantine agreement protocol and/or another suitable protocol. Once agreement has been reached, the assertion may be considered validated by the ledger nodes 104 and the submission may be recorded and/or otherwise entered into the ledger 102.

A querying system 106 interested in determining whether an assertion and/or a certain type of assertion (e.g., an assertion relating to a particular device and/or the like) has been recorded in the ledger 102 may be configured to query one or more of the ledger nodes 104 and/or other associated systems and receive associated responses. In various embodiments, the querying system 106 may query a system 110 (e.g., a derivative ledger node) maintaining a ledger derivative 108, which may comprise one of the ledger nodes 104 and/or another system, and may receive associated responses indicative of assertions recorded in the ledger derivative 108 and/or the ledger 102.

In certain embodiments, as illustrated, the ledger derivative 108 may be maintained by a separate derivative ledger node system 110, with entries in the derivative ledger 108 being generated based on entries and/or assertions recorded the ledger 102 and/or one or more other ledgers and/or information sources. In further embodiments, the ledger derivative 108 may be maintained by one or more of the ledger nodes maintaining the ledger 102. In various embodiments, entries in the ledger derivative 108 may be generated and/or otherwise recorded by the derivative ledger node system 110 based on entries in the ledger 102 in accordance with one or more policies and/or algorithms governing entries in the ledger derivative 108, which may be dependent, at least in part, on a type, application, and/or use of the ledger derivative 108. Among other things, use of a ledger derivative 108 consistent with various aspects of the disclosed embodiments may help streamline the validation of current state of information from the ledger 102 by performing various processing and/or analysis of entries in the ledger 102, allowing for information queries with reduced latency and/or providing useful conclusions and/or derivative information from the more granular data recorded in the ledger 102.

### Trusted Ledgers and Health Information Assertions

Consistent with various embodiments disclosed herein, one or more trusted ledgers may be used to record assertions relating to an individual's vaccination status. In certain, but not all, instances herein, an individual may be generally referred to as a user. A user may be associated with an account, which may be logically used in various aspects of the disclosed embodiments to represent the user. Although various examples described herein may relate to vaccination status determinations and/or associated information and/or assertions, it will be appreciated that aspects of the disclosed systems and methods may be applied and/or otherwise used in connection with a variety of other types of personal information which may, or may not, include personal health information such as vaccination status.

A variety of information may be included in an assertion relating to an individual's vaccination status. As described above, some assertions may be associative in nature. For example, an assertion may associate a key and/or a fingerprint of a key with an identifier of a user and/or an associated account, a service, an entity, and/or some type of authority, thereby creating an association between the identifier and the key. In further embodiments, an association reflected in an assertion may be between two entities. For example, an assertion may be associated with a user and/or an associated account, a service, an entity, and/or an authority with another user and/or account, service, entity, and/or authority. In some embodiments, such an association may be used to establish and/or otherwise determine trusted relationships between such entities.

In further embodiments, assertions may be associated with a user and/or an account and/or an associated account, a service, an entity, an authority with certain “fact” information. For example, a user and/or associated account may be associated with certain “fact” information comprising vaccination status information. Vaccination status information may comprise detailed vaccination status information such as, for example and without limitation, an indication that a particular vaccination was given to the user, an indication of a date that a user was administered a vaccine, and/or other related information (e.g., vaccine manufacturer, dosage information, lot number information, information relating to an individual and/or entity that administered the vaccination, and/or any other type of similar information).

In certain embodiments, vaccination status information may be generalized and/or derivative in nature. For example, vaccination status information may indicate that an individually is considered “fully vaccinated” without specifically referencing details regarding the administration of the vaccine dose (or doses) granting a user such a status. In some embodiments, vaccination status information may further reference to the standards and/or an authority setting the standards used to determine such a generalized and/or derivative indication of status (e.g., “fully vaccinated according to CDC standards,” “fully vaccinated according to CDC standards as of Sep. 10, 2022,” etc.). In various embodiments, this information may be used to determine what standards were used to define the more generalized and/or derivative vaccination status information at the time an associated assertion was made in a trusted ledger in the event that certain requirements associated with an individual's status are changed by a relevant authority (e.g., in the event that new booster doses become available and the standards for an individual being considered “fully vaccinated” change over time).

In some embodiments, generalized and/or derivative vaccination status information (e.g., “fully vaccinated,” etc.) may be maintained in one or more derivative TIDALs, which more granular assertions (e.g., details regarding the administration of the vaccine dose(s)) recorded in one or more other TIDALs. It will be appreciated that vaccination status information may be maintained in one or more TIDALs and/or derivative TIDALs in a variety of ways and/or configurations, which may depend on a particular application, context, and/or jurisdiction requirement(s), consistent with various aspects of the disclosed embodiments.

In yet further embodiments, an assertion may relate to and/or provide contextual information relating to other assertions entered into a ledger. For example, an assertion may not necessarily comprise external associations (e.g., an association between a user and key, a user and vaccination status information, etc.), but may provide contextual information that may be used to provide internal information relating to other entries in a ledger (and/or potentially other ledgers).

In some embodiments, an assertion may comprise an indication of criteria and/or a change in criteria used to generate other assertions included in a ledger. For example and without limitation, an assertion may indicate a criteria for an individual to be considered “fully vaccinated” (e.g., 2 primary doses of a vaccine plus a booster dose). Subsequent ledger entries may associate the individual (and/or an account associated with the individual) with a “fully vaccinated” vaccination status, and the earlier assertion may be examiner to determine what criteria were met for the individual to have been considered “fully vaccinated.”

In the event that the criteria for being considered “fully vaccinated” changes, another assertion may be entered into the ledger indicating the new criteria for an individual to be considered “fully vaccinated” (e.g., 2 primary dose, plus two booster doses). Ledger entries associating an individual with a “fully vaccinated” status preceding this entry may be determined to not necessarily meet the updated criteria for being considered “fully vaccinated.” Ledger entries associated an individual with “fully vaccinated” status following the entry detailing the updated criteria may be determined to meet the current vaccination status criteria. In various embodiments, when a definition and/or criteria of “fully vaccinated” and/or some other general assertion changes, derivative and/or more general assertions such as “Sakura is fully vaccinated” can be revoked, until such time as Sakura meets the new terms of “fully vaccinated”, in which case such an assertion may be re-recorded in a ledger. For example and without limitation, a new entry may be entered into a ledger that invalidates a prior entry indicating “fully vaccinated” status and/or a new entry may be entered indicating that the status for an individual is now “unvaccinated,” “partially vaccinated,” and/or another suitable designation.

### Verification of Vaccination Status

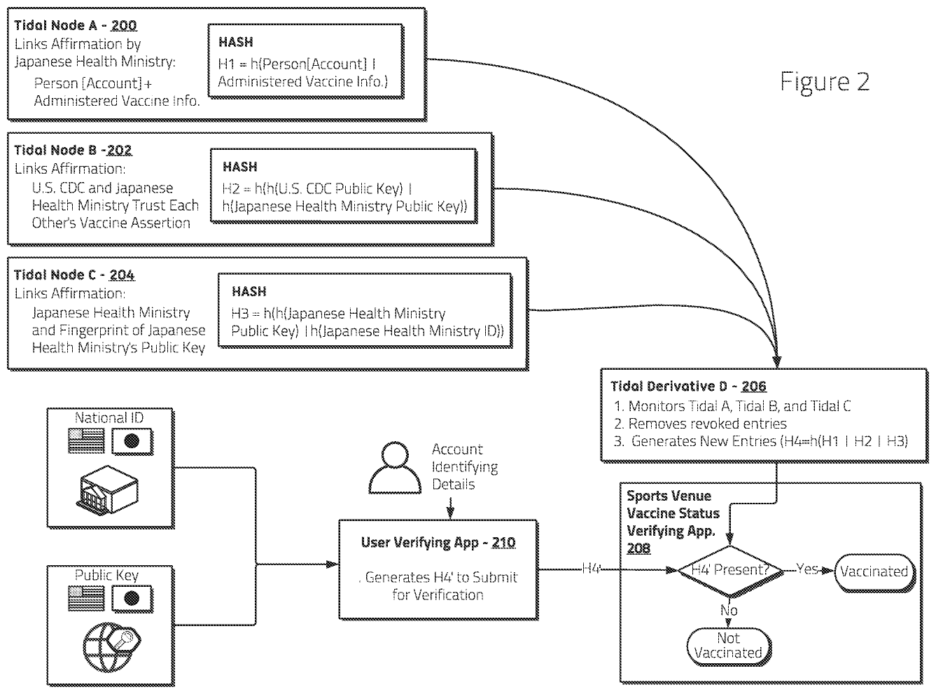

FIG. 2 illustrates a non-limiting example of an architecture for recording and validating vaccination status of an individual using a plurality of ledgers 200, 202, 204 consistent with certain embodiments disclosed herein. The architecture illustrated in FIG. 2 may comprise three trusted ledgers: ledgers A 200, B 202, and C 204. Although illustrated as TIDALs, will be appreciated that the ledgers may have all and/or a subset of the properties of a TIDAL described herein. The ledgers may be used to record the following assertions in the form of hash entries:
The Japanese Health Ministry asserts that an individual associated with a particular account is vaccinated.
The U.S. CDC and the Japanese Health Ministry trust one another's assertions regarding vaccine administration.
The Japanese Health Ministry is an authenticated authority.

For example, TIDAL A 200 may record an assertion associating an account (e.g., an account associated with a subject individual) with vaccination status information. The vaccination status information may comprise detailed vaccination status information (e.g., information regarding specific doses, administration dates, lot numbers, etc.) and/or more generalized vaccination status information (e.g., “fully vaccinated according to current criteria” or the like). The assertion may comprise a hash—H1—calculated based on the account information and/or the vaccination status information. For example and without limitation, in the illustrated example, the assertion may comprise a hash of a concatenation of an account information identifier and the vaccination status information.

TIDAL B 202 may record an association indicating that the U.S. CDC and the Japanese Heath Ministry trust one another's assertions regarding vaccine administration and/or vaccination status. In some embodiments, such an assertion may comprise a hash—H2—calculated based on unique information associated with the relevant authorities such as, for example and without limitation, public keys associated with the authorities and/or fingerprints thereof. For example and without limitation, in the illustrated example, the assertion may comprise a hash of a concatenation of a hash of a public key associated with the U.S. CDC and a hash of a public key associated with the Japanese Health Ministry.

TIDAL C 204 may record an assertion that allows for authentication of one or more authorities. In some embodiments, such an assertion may securely associate a key and/or fingerprint thereof associated with an authority with a unique identifier associated with the authority. In certain embodiments, the assertion may comprise a hash—H3—calculated based a public key associated with an authority and a unique identifier associated with the authority. For example and without limitation, in the illustrated example, the assertion may comprise a hash of a concatenation of a hash of a public key associated with the Japanese Health Ministry with a hash of a unique identifier associated with the Japanese Health Ministry.

A derivative ledger—TIDAL derivative D 206—may monitor TIDAL A 200, TIDAL B 202, and/or TIDAL C 204 and generate and/or record new entries based on the monitored ledgers 200-204. TIDAL derivative D 206 may further remove revoked entries in the monitored ledgers by generating and recording new entries indicative of the same (e.g., via inclusion of an entry in TIDAL derivative D 206 indicating that a particular entry in one of the monitored ledgers 200-204 was revoked).

Entries in TIDAL derivative D 206 may comprise a hash value—H4—calculated based on entries in the monitored ledgers 200-204. For example and without limitation, in the illustrated example, the assertion may comprise a hash of a concatenation of entries recorded in the monitored ledgers (e.g., a concatenation of H1, H2, and H3).

Based on the entries included in the TIDALs 200-204 and/or the TIDAL derivative 206, an individual may authenticate their vaccination status with a gatekeeper service (e.g., a gatekeeper service associated with a particular venue, location, security and/or administration checkpoint, and/or the like) in a trusted manner. In some embodiments, such an authentication process may involve a user device and a gatekeeper service device executing respective verification applications 208, 210.

In certain embodiments, based on available information from one or more sources, which in some embodiments may include the TIDALs 200-204, and in further embodiments may include one or more other sources (e.g., personal and/or governmental databases and/or registries), a user application 210 executing one a user device, which in certain instances herein may be referred to as a passport application, vaccination passport application, verification application, and/or derivatives thereof, may provide certain information to a gatekeeping service application 208 executing on a gatekeeping service device and/or system. The gatekeeping service application 208 may verify the information received from the user application 208 based on information accessed from the TIDALs 200-204 and/or the TIDAL derivative 206 (and/or one or more other ledgers) to authenticate a status and/or other information proffered by the user (e.g., vaccination status and/or the like).

In the illustrated non-limiting example, a user may arrive at a sports venue to attend a match. To gain entry to the venue, the user may open a user application 210 executing on a mobile device to verify their vaccination status with a gatekeeping service. Using a camera included in and/or otherwise associated with her device, the user may capture certain identifying details (e.g., by photographic capture of a national identification card). Additionally and/or alternatively, the identifying details may be captured by the mobile device using another method (e.g., downloaded and/or entered by a user). It will be appreciated that a wide variety of identification details may be used in connection with various aspects of the disclosed embodiments.

Using the user application 210, the user may capture and/or otherwise receive and/or access one or more of (1) identifying details that were presented to the Japanese Ministry of Health when they were vaccinated, (2) identification information associated with the Japanese Ministry of Health recorded when they received their vaccination and/or downloaded from a web address and/or other location associated with the Japanese Ministry of Health, and (3) the public key of the Japanese Ministry of Health. In some embodiments, this type of information and/or a subset thereof may be provided by a user to the user application 210 as account identifying details. In further embodiments, the user application 210 may use information provided by the user to access certain information from one or more sources, which may be managed by a user device and/or one or more remote systems and/or services accessible by the user device and/or the user application 210 (e.g., public key databases and/or information services, identification information sources, etc.). Based on the received information, the user application 210 may calculate a hash “candidate” H4′.

The user device may present to and/or otherwise communicate the hash candidate H4′ to a gatekeeping application 208 executing on a gatekeeping service device and/or system associated with the sports venue operating as a vaccine status verification application. The gatekeeping application 208 and/or an associated system and/or device may query the TIDAL derivative D 206 to determine whether the hash candidate is recorded in the derivative ledger. Alternatively and/or additionally, the gatekeeping 208 may query one or more of the TIDALs 200-204 directly to determine whether assertions that may be used to generate the hash candidate H4′ are present in the TIDALs 200-204.

If the hash candidate is present in the TIDAL derivative D 206, the gatekeeping application 208 may determine that the user's vaccinations status has been validated. Otherwise, it may be determined that the user is not vaccinated and/or that their vaccination status has not been recorded and/or otherwise documented by the associated authorities in the TIDALs 200-204 and/or TIDAL derivative 206.

As described above, the user application 210 may generate candidate hash H4′ and provide the generated candidate hash to the gatekeeping application 208 for verification. In some embodiments, there may be a shared protocol, format, and/or method of generating candidate hashes for verification purposes known by the user application 210, the gate keeping application 206, and/or services and/or authorities managing the one or more TIDALs 200-204 and/or TIDAL derivative 206.

In further embodiments, the user application 210 may not immediately be aware of the protocol, format, and/or method for generating candidate hashes, but may receive such information from the gatekeeping application 208. For example and without limitation, in some embodiments, during an initialization and/or handshake process between the user application 210 and the gatekeeping application 208, the gatekeeping application 208 may exchange certain details regarding how the user application 210 should generate candidate hash values submitted to the gatekeeping application 408 for verification and/or the constituent information that should be used to generate such hash values.

In further embodiments, the user application 210 may not generate the candidate hash itself, but may communicate constituent information that may be used to generate the candidate hash to the gatekeeping application 208. Based on this information, the gatekeeping application 208 may generate the candidate hash, checking it against the one or more TIDALs 200-204 and/or TIDAL derivative 206 for verification. In some embodiments, the gatekeeping application 208 may communicate certain details to the user application 210 regarding the information it may need to compute a candidate hash (e.g., in a request message and/or the like), and the user application 210 may submit the requested information to the gatekeeping application 210 as part of the verification process.

In the example described above in relation to FIG. 2, a simplified case of mutual trust between the applicable governmental agencies in U.S. and Japan was used for purposes of illustration and explanation. It will be appreciated, however, that more complex trust relationships may also be expressed within the scope of various embodiments of the disclosed techniques. In some embodiments, for example, TIDAL B 202 may be configured to record assertions expressing trust relationships as independent (e.g., unitary and/or directional) trust assertions. For example and without limitation, a value H2=h(h(Japanese Health Ministry public key|U.S. CDC public key)) may be express the relationship that “Japanese Health Ministry trusts the U.S. CDC”, while a value h(h(U.S. CDC public key|Japanese Health Ministry public key)) may be understood to express the relationship “U.S. CDC trusts the Japanese Health Ministry.” In this manner, trust relationships may be maintained independently from one another.

TIDAL B 202 and potentially its associated derivatives (e.g., TIDAL derivative D 206) may be configured to update such associations in a date and/or entry ordered fashion to reflect the current status of trust between entities. As conditions change and/or more information becomes known about policies and/or procedures associated with assertions by certain entities, various entities in the system can revoke and/or update their trust assertions accordingly. Other trusted ledgers and/or derivative ledgers derivatives may query the ledgers 200-206 periodically to determine the latest trust status as may be appropriate for their application.

### Verification of Gatekeeper Services

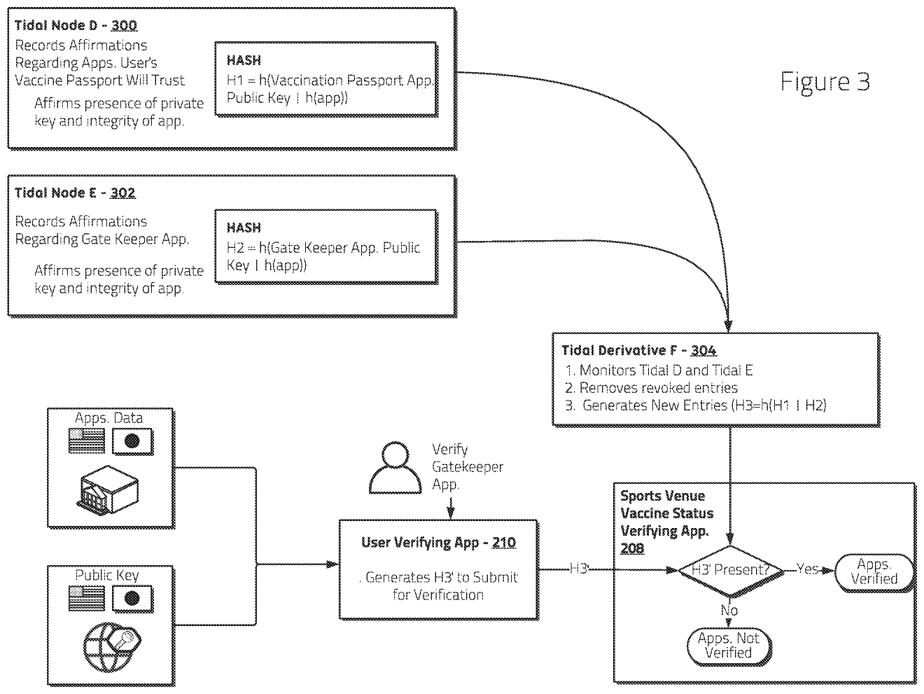

FIG. 3 illustrates another non-limiting example of an architecture for authenticating a gatekeeping application using a plurality of ledgers consistent with certain embodiments disclosed herein. As described above, various embodiments disclosed herein may operate to establish trust between interested parties. Consistent with embodiments disclosed herein, trusted ledgers and/or derivative ledgers (e.g., TIDALs 300, 302 and/or TIDAL derivatives 304) may provide online and/or offline verification of application credentials to ensure that the user application 210 (and/or an associated device) is trusted by a gatekeeping application 208 and/or an associated device and/or system (which in certain embodiments may be associated with a managed venue), and that the user application 210 (and/or an associated device) may trust the gatekeeping application 208, device, and/or system.

In the illustrated non-limiting example, a first trusted ledger—TIDAL D 300—may record an assertion associating a public key associated with a user application 210 and a fingerprint of the user application 210 such as, for example and without limitation, a hash of the user application 210. In some embodiments, the assertion may comprise a hash—H1—generated based on the public key associated with a user application 210 and the fingerprint of the user application 210. For example and without limitation, as illustrated, a hash may be generated of a concatenation of the public key associated with a user application 210 and a hash of the user application 210.

A second trusted ledger—TIDAL E 302—may record an assertion associating a public key associated with a gatekeeping application 208 and a fingerprint of the gatekeeping application 208 such as, for example and without limitation, a hash of the gatekeeping application 208. In some embodiments, the assertion may comprise a hash—H2—generated based on the public key associated with a gatekeeping application 208 and the fingerprint of the gatekeeping application 208. For example and without limitation, as illustrated, a hash may be generated of a concatenation of the public key associated with a gatekeeping application 208 and a hash of the gatekeeping application 208.

A trusted derivative ledger—TIDAL derivative F 304—may record assertions generated based on the first and second ledgers 300, 302 in a relatively high-performance hash table, which in some embodiments may be distributed. For example, TIDAL derivative F 304 may monitor TIDAL D 300 and TIDAL E 302 and generate and/or record new entries based on the monitored ledgers 300, 302. TIDAL derivative F 304 may further remove revoked entries in the monitored ledgers by generating and recording new entries indicative of the same (e.g., via inclusion of an entry in TIDAL derivative F 304 indicating that a particular entry in one of the monitored ledgers 300, 302 was revoked).

Entries in TIDAL derivative F 304 may, in some embodiments, comprise a hash value—H3—calculated based on entries in the monitored ledgers 300, 302. For example and without limitation, in the illustrated example, the assertion may comprise a hash of a concatenation of entries recorded in the monitored ledgers (e.g., a concatenation of H1 and H2).

Consistent with various embodiments disclosed herein, the user application 210 and/or the gatekeeping application 208 and/or associated devices and/or systems may compute assertions and/or hashes of assertions based on the applications and/or associated and check whether the computed assertions and/or hashes thereof are included in the trusted derivative ledger 304 (and/or the other trusted ledgers 300, 302), thereby providing an indication of trust of the applications 208, 210. For example and without limitation, as illustrated, the user application 210 may calculate a hash candidate H3′ based, at least in part, on public key information associated with the user application 210 and/or the gatekeeper application 208 and/or hashes of the respective applications.

The user device may present to and/or otherwise communicate the hash candidate H3′ to the gatekeeping application 208 executing on a gatekeeping service device and/or system associated with the sports venue. The gatekeeping application 208 and/or an associated system and/or device may query the TIDAL derivative F 304 to determine whether the hash candidate is recorded in the derivative ledger. Alternatively and/or additionally, the vaccine status application 304 may query one or more of the TIDALs 300, 302 directly to determine whether assertions that may be used to generate the hash candidate H3′ are present in the TIDALs 300, 302.

If the hash candidate is present in the TIDAL derivative F 304, the gatekeeping application 208 may determine that the user application 210 is valid and/or otherwise authenticated. Similarly, the user application 210 may determine that the gatekeeping application 208 is valid and/or otherwise authenticated.

### Other Personal Health Information Assertions

Although many of the examples described previously have focused on assertions regarding vaccination status of a user, various embodiments described herein may also be applied to other forms of data. For example and without limitation, assertions may also be recorded in a trusted ledger regarding verified results of an individual's virus and/or antibody test results. In the early days of an epidemic or pandemic, viable and/or approved vaccines may not yet be widely available. Through scientific study, however, it may be determined that individuals that have tested positive for the virus and have recovered and/or have tested positive for a threshold level of antibodies to the virus are sufficiently protected against the virus and/or do not present a substantial risk of transmission/re-transmission of the virus. That is, an individual may not necessarily be vaccinated, but may nevertheless exhibit a measure of immunity based on prior infection.

In such instances, a trusted ledger may be established to record assertions regarding test status associated with individuals. The assertions may, for example and without limitation, include assertions associating an individual name and test result, and may include and/or reference associated information describing the type of test, date of test, serial and/or lot number of the test, individual and/or organization performing the test, and/or other information related to test administration or processing. In some embodiments, one or more trusted ledger derivatives may periodically scan the data and provide a determination of immunity and/or transmissibility risk associated with an individual based on a current policy in effect. The policy used for such determination may incorporate a variety of input factors including but not limited to elapsed time since a test result, type of test, number of tests performed with consistent results, and/or levels of antibodies detected.

The policies used by trusted ledgers or ledger derivatives to determine immunity and/or vaccination status may be dynamically updated, and their contents may be updated accordingly. For example, a policy for determining vaccination status, immunity status, and or transmissibility risk, may be modified as new information and/or factors are discovered. Consistent with embodiments disclosed herein (and as discussed in more detail above), such policy changes may be recorded in the ledger, providing contextual information relating to the ledger entries preceding and/or following an entry detailing a policy change.

In another non-limiting example, a public health policy may be updated based on the discovery of a new virus variant that was not previously tested or identified by a test. Similarly, it may be determined that the levels of antibodies diminish over time and no longer provide sufficient immunity after a certain amount of time since a positive test (or a vaccination). In yet another example, a new variant may be discovered to be spreading within a community for which certain vaccines are found to retain a sufficient level of protection, while others do not. In such instances, a ledger derivative may re-scan the appropriate ledger instance(s) (e.g., with the test results, vaccination history, etc.) using the updated policy, and update the associated status for the individuals in the derivative database ledger. Alternatively, or in addition, a new ledger derivative with the new policy may be created and may serve as the trusted source for status based on the new criteria.

System architectures consistent with certain embodiments described herein may allow for rapid wide-scale deployment in a decentralized manner, flexible adaptation of policies as new immunological and/or epidemiological information becomes available, description of trust relationships by entities so that verifying entities may reliably and efficiently evaluate the status of individuals, and/or the like. Certain system architectures using the techniques described herein may allow “front-line” entities to record assertions in a ledger according to their scope of responsibility and authority such as, for example and without limitation: Individual A received vaccination X on date Y, or Individual B tested positive for test T on date D. Based on available scientific data, epidemiological knowledge, and other public health considerations, trusted authorities (e.g. governmental authorities, medical authorities, public health authorities, etc.) may establish their own policies to use this data to determine associated risk factors and record digitally signed assertions in a trusted ledger (or ledger derivative) relating to an individual's epidemiological risk status. Using certain techniques described herein, verifying entities may in some implementations confirm trust relationships between the parties making such assertions and confidently use the assertions to make rapid operational decisions at scale and without the need to reveal an undue amount of personal information to the individuals or systems involved in the verification process.

### Assertion Type Information

In various embodiments, facts included in assertions recorded to ledgers may not be limited to a simple string, including those where one or more public key hashes are embedded. In certain embodiments, facts can be tagged with an assertion type, a standardized identifier that describes the semantics of the fact statement. This may allow for multiple different types of assertions to be stored in a same TIDAL without ambiguity. One non-limiting example of a possible implementation is to serialize JSON objects, including both the assertion type and the fact, into a string, and then take the hash of that string as the entry into the TIDAL.

As a non-limiting example, consider the following two facts: “CompanyA TRUSTS CompanyB” and “CompanyA OWNS CompanyB”. These are different facts. Simply representing these as “h(pub_key_of_companya)|h(pub_key_of_companyb)” may not provide particularly meaningful distinctions of “trusts” versus “owns”. This ambiguity can be resolved by using two distinct TIDALs to store the corresponding assertions. However, when there are many different fact types, it is not always efficient to create new TIDALs to store these multiple different kinds of facts. Thus, representing the facts as, for example, “TRUSTS|h(pub_key_of_company_a)|h(pub_key_of_companyb)” and “OWNS|h(pub_key_of_company_a)|h(pub_key_of_companyb)”, may allow the assertions for these facts (e.g., hashes of these strings) to be distinguished from one another.

Another representation of these tagged facts is through the use of JSON encoding. For example and without limitation

{
“type”: “TRUSTS”,
“fact”: “h(pub_key_of_company_a) | h(pub_key_of_company_b)”
}

The string encoding of this JSON object includes the “assertion type” (e.g., fact type), reducing any ambiguity—assuming the assertion types are themselves standardized for a given TIDAL.

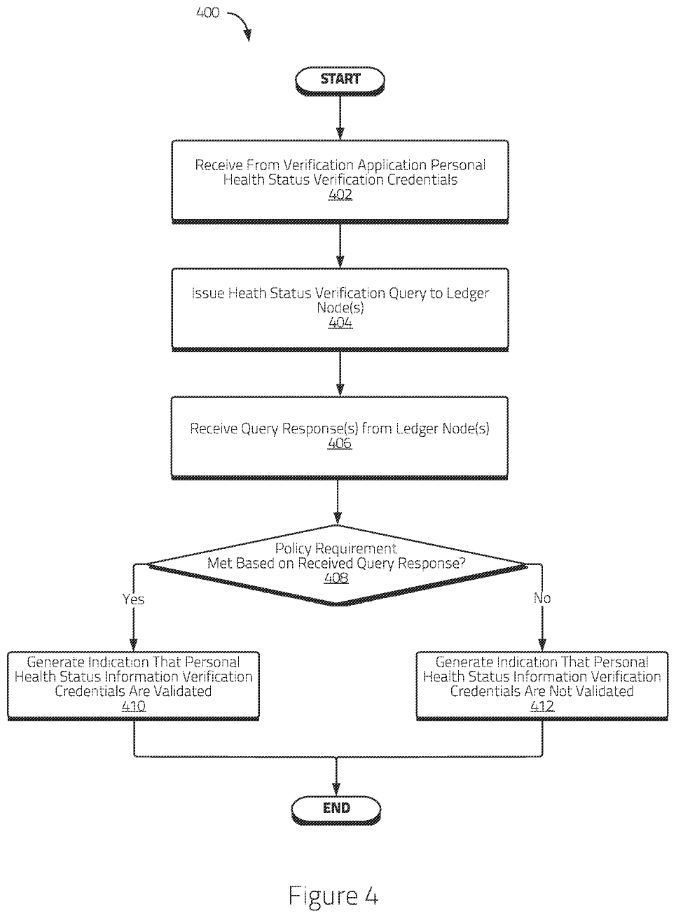

FIG. 4 illustrates a flow chart of a non-limiting example of a method 400 for verifying personal health status information associated with a user consistent with certain embodiments of the present disclosure. The illustrated method 400 may be implemented in a variety of ways, including using software, firmware, hardware, and/or any combination thereof. In certain embodiments, various aspects of the method 400 and/or its constituent steps may be performed by system and/or device and/or an associated gatekeeping application and/or personal health status verification application, and/or any other suitable application, device, system and/or service or combination of applications, devices, systems, and/or services.

At 402, a gatekeeping application executing on a system may receive personal health status information verification credentials from a verification application executing on a user device associated with a user. Consistent with embodiments disclosed herein, the personal health status information verification credentials may comprise a variety of information. For example and without limitation, the personal health status information verification credentials may comprise one or more of an indication of identification information associated with the user, an indication of health status information associated with the user, an indication of a first authority associated with the health status information, and an indication of a second authority associated with the system.

In various embodiments, the identification information associated with the user may comprise an account associated with the user, although other types of user identification information and/or credentials may also be used. The health status information associated with the user may comprise a variety of types of information. For example and without limitation, the health status information associated with the user may comprise one or more of an indication of a vaccination status associated with the user, an indication of at least one administered vaccine dose associated with the user, a vaccine administration date associated with at least one administered vaccine dose, a vaccine manufacturer associated with at least one administered vaccine dose, vaccine lot information associated with at least one administered vaccine dose, vaccine dosage information data associated with at least one administered vaccine dose, identification information relating to an individual that administered at least one administered vaccine dose, and identification information relating to an entity associated with the administration of the at least one administered vaccination dose.

In further embodiments, the health status information associated with the user may comprise an indication of an immunity status associated with the user which may, or may not, depend on a vaccination status of the user. For example and without limitation, in some embodiments, health status information associated with the use the indication of the immunity status associated with the user may comprise one or more of an infection test result associated with the user and an antibody test result associated with the user.

In certain embodiments, the first authority and second authority may comprise public health authorities (e.g., local health departments, the U.S. CDC, various health ministries, etc.) that, in some embodiments, may be different health authorities. For example, the first authority may comprise a heath authority responsible for a jurisdiction in which a user has received a vaccination, and the second authority may comprise a public health authority associated with the gatekeeping application and/or an associated managed location. As detailed above, the health status information associated with the user may comprise an indication of a vaccination status associated with the user and the first authority may comprise a public health authority associated with the indication of the vaccination status (e.g., a health authority that determined and/or otherwise set policy and criteria underlying the indication of the vaccination status).

In certain embodiments, the gatekeeping application may communicate certain protocol requirements and/or details to the user device, system, and/or associated application relating to information that should be provided to the gatekeeping application for the gatekeeping application to verify whether a user is permitted access to a managed location. For example, in some embodiments, the gatekeeping application may communicate to a user application details regarding what information should be included in personal health status information verification credentials submitted to the gatekeeping application for verification, a protocol and/or method for generating such credentials and/or a subset thereof, and/or the like.

The information included in the personal health status information verification credentials may be received in a variety of forms and/or may be represented in a variety of ways consistent with various aspects of the disclosed embodiments. For example, in some embodiments, the information associated with the user and the indication of health status information may be represented by a first hash included in the personal health status information verification credentials. In certain embodiments, the first hash may be generated based on the indication of identification information associated with the user and the indication of health status information.

The indication of the first authority and/or the indication of the second authority may also be received in a variety of forms and/or may be represented in a variety of ways consistent with various disclosed embodiments. For example, in some embodiments, the indication of the first authority may comprise a public key associated with the first authority and/or a fingerprint thereof (e.g., a hash of the public key). Similarly, the indication of the second authority may comprise a public key associated with the second authority and/or a fingerprint thereof (e.g., a hash of the public key). In certain embodiments, the indication of the first authority and/or the indication of the second authority may be used to generate aspects of a verification request that may be used to determine whether the first authority is trusted by the second authority and, similarly, whether the second authority is trusted by the first authority.

In some embodiments, the indication of the first authority and/or the indication of the second authority may be represented by a second hash included in the personal health status information verification credentials. Consistent with certain embodiments disclosed herein, the second hash may comprise a hash generated based on a third hash of the public key associated with the first authority and a fourth hash of the public key associated with the second authority.

A health status verification query may be generated at 404 and issued to one or more ledger nodes maintaining one or more trusted distributed assertion ledgers. The health status verification query may be generated based the received personal health status information verification credentials, potentially by processing the credentials. In some embodiments, the personal health status information verification credentials may comprise a hash value generated based on a hash of the indication of identification information associated with the user and the indication of health status information associated with the user and a hash of the indication of a first authority associated with the health status information and the indication of a second authority associated with the system. This received hash may be issued to the one or more ledger nodes as part of the health status verification query.

In further embodiments, the constituent information used to generate the hash may be received by the gatekeeping application, which may generate the hash and issue it as part of the query to the one or more ledger nodes. For example, the gatekeeping application may generate a hash based on the received indication of identification information associated with the user, the indication of health status information associated with the user, the indication of a first authority associated with the health status information, and the indication of a second authority associated with the system.

In some embodiments, the one or more trusted distribution assertion ledgers may comprise cryptographically linked ledger entries, which may comprise one or more blockchain ledgers. In further embodiments, at least one of the one or more trusted distribution assertion ledgers may comprise a derivative ledger. That is, at least one trusted distributed assertion ledger of the one or more trusted distributed assertion ledgers may comprise one or more entries derived from at least subset of entries of one or more other trusted ledgers. In some embodiments, the health status verification query may comprise at least one indication of an assertion type associated with the health status verification query.

At 406, one or more query responses may be received from the one or more ledger nodes. In certain embodiments, the one or more query responses may indicate whether certain information (e.g., hash values and/or the like) included in the health status verification query has been included and/or otherwise recorded in the queried ledgers.

Based on the received query response, it may be determined at 408 whether the personal health status information verification credentials meet at least one policy requirement enforced by the gatekeeping application. For example, the at least one policy requirement may require that the query response indicate that information included in the health status verification query issued to the ledger node(s) has been recorded in one or more of the associated queried ledgers, which may indicate that a user meets threshold vaccination and/or immunity status for a location and/or venue managed by a gatekeeping service. If the at least one policy requirement has been met, an indication may be generated at 410 indicating that the personal health status information verification credentials provided by the user device are validated. If the at least one policy requirement has not been met, an indication may be generated at 412 indicating that the personal health status information verification credentials provided by the user device were not validated.

A variety of indications may be used in connection with various disclosed embodiments. For example, the indication may comprise an indication provided by and/or otherwise issued via a user interface of the system. The indication may comprise, for example, a text indication, a visual indication, an audio indication, and/or the like. In further embodiments, the indication may be communicated to one or more other devices that may, for example, include the user device and/or another device and/or system associated with the gatekeeping service.

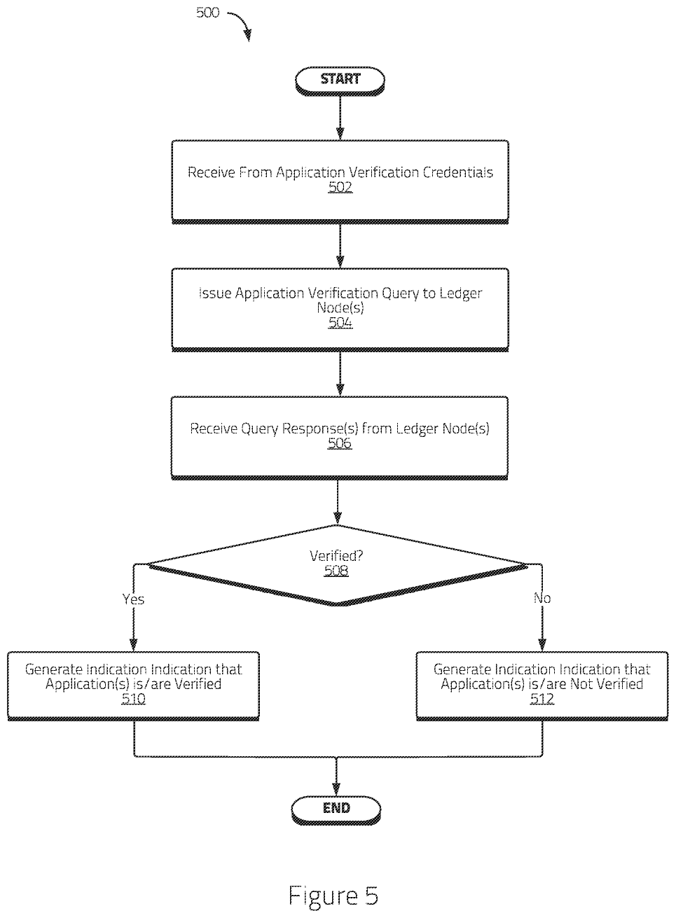

FIG. 5 illustrates a flow chart of a non-limiting example of a method for authenticating a user application consistent with certain embodiments of the present disclosure. The illustrated method 500 may be implemented in a variety of ways, including using software, firmware, hardware, and/or any combination thereof. In certain embodiments, various aspects of the method 500 and/or its constituent steps may be performed by system and/or device and/or an associated gatekeeping application and/or personal health status verification application associated with a user device, and/or any other suitable application, device, system and/or service or combination of applications, devices, systems, and/or services.

At 502, a gatekeeping application executing on a system may receive application verification credentials from a verification application executing on a user device. In some embodiments, application verification credentials may be received from a trusted operating system of the user's device and/or system that may verify and/or provide some indication of verification that the application has not been tampered with and/or that associated credentials are valid and/or otherwise associated with the application.

The application verification credentials may comprise an indication associated with the verification application. In some embodiments, the indication associated with the verification application may comprise and/or otherwise be generated based on a public key associated with the verification application and/or a fingerprint thereof (e.g., a hash of the public key) and/or a unique fingerprint of the verification application (e.g., a hash of the application). For example and without limitation, the application verification credentials may comprise a hash generated based on a hash of a public key associated with the verification application and a hash of the application itself.

The gatekeeping application may issue an application verification query to one or more ledger nodes at 504. In some embodiments, the application verification query may be generated based, at least in part, on the application verification credentials received from the verification application. The application verification query may further comprise an indication associated with the gatekeeping application verification credentials, which may be received from the verification application executing on the user device as part of the application verification credentials and/or be generated by the gatekeeping application itself. In some embodiments, the gatekeeping application verification credentials may be generated similar to the indication associated with the application verification credentials. For example and without limitation, the gatekeeping application verification credentials may comprise a hash generated based on a hash of a public key associated with the gatekeeping application and a hash of the gatekeeping itself.

At 506, one or more query responses may be received from the one or more ledger nodes. In certain embodiments, the one or more query responses may indicate whether certain information (e.g., hash values and/or the like) included in the application verification query has been included and/or otherwise recorded in the queried ledgers. Based on the received responses, corresponding indications may be generated at either 508 or 510.

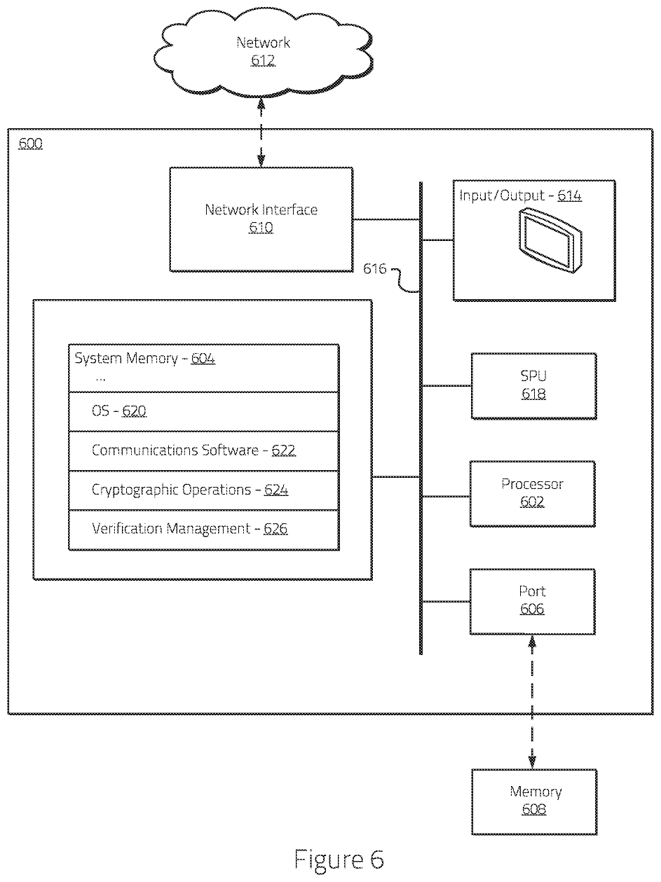

FIG. 6 illustrates a non-limiting example of a system 600 that may be used to implement certain embodiments of the systems and methods of the present disclosure. The system 600 may comprise a variety of computing devices and/or systems, including any computing system suitable to implement the systems and methods disclosed herein. In various embodiments, the system 600 may comprise a system and/or device associated with a user and/or querying system, an assertion submitter, a witness, trusted ledger node operator and/or trusted ledger management system, a user device and/or system, a gatekeeping device and/or system, and/or any other service, system, device, entity, node, application and/or component configured to implement aspects of the embodiments of the disclosed systems and methods.

The various systems and/or devices used in connection with aspects the disclosed embodiments may be communicatively coupled using a variety of networks and/or network connections (e.g., network 612). In certain embodiments, the network 612 may comprise a variety of network communication devices and/or channels and may utilize any suitable communications protocols and/or standards facilitating communication between the systems and/or devices. The network 612 may comprise the Internet, a local area network, a virtual private network, and/or any other communication network utilizing one or more electronic communication technologies and/or standards (e.g., Ethernet or the like). In some embodiments, the network 612 may comprise a wireless carrier system such as a personal communications system (“PCS”), and/or any other suitable communication system incorporating any suitable communication standards and/or protocols. In further embodiments, the network 612 may comprise an analog mobile communications network and/or a digital mobile communications network utilizing, for example, code division multiple access (“CDMA”), Global System for Mobile Communications or Groupe Special Mobile (“GSM”), frequency division multiple access (“FDMA”), and/or time divisional multiple access (“TDMA”) standards. In certain embodiments, the network 612 may incorporate one or more satellite communication links. In yet further embodiments, the network 612 may utilize IEEE's 802.11 standards, Bluetooth®, ultra-wide band (“UWB”), Zigbee , and or any other suitable standard or standards.

The various systems and/or devices used in connection with aspects of the disclosed embodiments may comprise a variety of computing devices and/or systems, including any computing system or systems suitable to implement the systems and methods disclosed herein. For example, the connected devices and/or systems may comprise a variety of computing devices and systems, including laptop computer systems, desktop computer systems, server computer systems, distributed computer systems, smartphones, tablet computers, and/or the like.

In certain embodiments, the systems and/or devices may comprise at least one processor system configured to execute instructions stored on an associated non-transitory computer-readable storage medium. As discussed in more detail below, systems used in connection with implementing various aspects of the disclosed embodiments may further comprise a secure processing unit (“SPU”) configured to perform sensitive operations such as trusted credential and/or key management, cryptographic operations, secure policy management, and/or other aspects of the systems and methods disclosed herein. The systems and/or devices may further comprise software and/or hardware configured to enable electronic communication of information between the devices and/or systems via a network using any suitable communication technology and/or standard.

As illustrated in FIG. 6, the example system 600 may comprise: a processing unit 602; system memory 604, which may include high speed random access memory (“RAM”), non-volatile memory (“ROM”), and/or one or more bulk non-volatile non-transitory computer-readable storage mediums (e.g., a hard disk, flash memory, etc.) for storing programs and other data for use and execution by the processing unit 602; a port 606 for interfacing with removable memory 608 that may include one or more diskettes, optical storage mediums (e.g., flash memory, thumb drives, USB dongles, compact discs, DVDs, etc.) and/or other non-transitory computer-readable storage mediums; a network interface 610 for communicating with other systems via one or more network connections and/or networks 612 using one or more communication technologies; a user interface 614 that may include a display and/or one or more input/output devices such as, for example, a touchscreen, a keyboard, a mouse, a track pad, and the like; and one or more busses 616 for communicatively coupling the elements of the system.

In some embodiments, the system 600 may, alternatively or in addition, include a trusted execution environment and/or an SPU 618 that is protected from tampering by a user of the system or other entities by utilizing secure physical and/or virtual security techniques. A trusted execution environment and/or a SPU 618 can help enhance the security of sensitive operations such as personal information management, trusted credential and/or key management, privacy and policy management, and other aspects of the systems and methods disclosed herein. In certain embodiments, the trusted execution environment and/or SPU 618 may operate in a logically secure processing domain and be configured to protect and operate on secret information, as described herein. In some embodiments, the trusted execution environment and/or a SPU 618 may include internal memory storing executable instructions or programs configured to enable the SPU 618 to perform secure operations, as described herein.

The operation of the system 600 may be generally controlled by the processing unit 602 and/or an SPU 618 operating by executing software instructions and programs stored in the system memory 604 (and/or other computer-readable media, such as memory 608, which may be removable). The system memory 604 may store a variety of executable programs or modules for controlling the operation of the system. For example, the system memory may include an operating system (“OS”) 620 that may manage and coordinate, at least in part, and/or system hardware resources and provide for common services for execution of various applications.

The system memory 604 may further include, without limitation, communication software 622 configured to enable in part communication with and by the system, one or more applications, a cryptographic operation module 624 configured to perform various aspects of the disclosed embodiments (e.g., cryptographic key and hash generation operations, key management operations, etc.), one or more verification management modules 626 configured to perform various aspects of the user health status and/or personal information verification processes and/or application verification processes disclosed herein, and/or any other information, modules, and/or applications configured to implement embodiments of the systems and methods disclosed herein.

The systems and methods disclosed herein are not inherently related to any particular computer, electronic control unit, or other apparatus and may be implemented by a suitable combination of hardware, software, and/or firmware. Software implementations may include one or more computer programs comprising executable code/instructions that, when executed by a processor, may cause the processor to perform a method defined at least in part by the executable instructions. The computer program can be written in any form of programming language, including compiled or interpreted languages, and can be deployed in any form, including as a standalone program or as a module, component, subroutine, or other unit suitable for use in a computing environment. Further, a computer program can be deployed to be executed on one computer or on multiple computers at one site or distributed across multiple sites and interconnected by a communication network.

Software embodiments may be implemented as a computer program product that comprises a non-transitory storage medium configured to store computer programs and instructions, that when executed by a processor, are configured to cause the processor to perform a method according to the instructions. In certain embodiments, the non-transitory storage medium may take any form capable of storing processor-readable instructions on a non-transitory storage medium. A non-transitory storage medium may be embodied by a compact disk, digital-video disk, a magnetic disk, flash memory, integrated circuits, or any other non-transitory digital processing apparatus memory device.

Although the foregoing has been described in some detail for purposes of clarity, it will be apparent that certain changes and modifications may be made without departing from the principles thereof. It should be noted that there are many alternative ways of implementing both the systems and methods described herein. Accordingly, the present embodiments are to be considered as illustrative and not restrictive, and the invention is not to be limited to the details given herein but may be modified with the scope and equivalents of the appended claims.

What is claimed is:

1. A method for verifying personal health status information performed by a gatekeeping application executing on a system comprising a processor and a non-transitory computer-readable storage medium storing instructions that, when executed by the processor, cause the system to perform the method, the method comprising:
receiving, by the gatekeeping application from a verification application executing on a user device associated with a user, personal health status information verification credentials, the personal health status information verification credentials comprising:
an indication of identification information associated with the user,
an indication of health status information associated with the user,
an indication of a first authority associated with the health status information, and
an indication of a second authority associated with the system;
issuing a health status verification query to one or more ledger nodes maintaining one or more trusted distributed assertion ledgers, the health status verification query being generated based on the received personal health status information verification credentials;
receiving, from the one or more ledger nodes, at least one query response generated by the one or more ledger nodes,
determining, based on the received query response, whether the personal health status information verification credentials meet at least one policy requirement enforced by the gatekeeping application; and
generating, based on the determination, an indication of whether the personal health status information verification credentials are validated by the gatekeeping application.

2. The method of claim 1, wherein the identification information associated with the user comprises an account associated with the user.

3. The method of claim 1, wherein the health status information associated with the user comprises an indication of a vaccination status associated with the user.

4. The method of claim 1, wherein the health status information associated with the user comprises an indication of at least one administered vaccine dose associated with the user.

5. The method of claim 4, wherein the health status information further comprises one or more of a vaccine administration date associated with the at least one administered vaccine dose, a vaccine manufacturer associated with the at least one administered vaccine dose, vaccine lot information associated with the at least one administered vaccine dose, vaccine dosage information data associated with the at least one administered vaccine dose, identification information relating to an individual that administered the at least one administered vaccine dose, and identification information relating to an entity associated with the administration of the at least one administered vaccination dose.

6. The method of claim 1, wherein the health status information associated with the user comprises an indication of an immunity status associated with the user.

7. The method of claim 6, wherein the indication of the immunity status associated with the user comprises one or more of an infection test result associated with the user and an antibody test result associated with the user.

8. The method of claim 1, wherein the first authority and second authority comprise public health authorities.

9. The method of claim 1, wherein the health status information associated with the user comprises an indication of a vaccination status and the first authority comprises a first public health authority associated with the indication of the vaccination status.

10. The method of claim 1, wherein the system is associated with a managed location.

11. The method of claim 10, wherein the second authority comprises a public health authority associated with the managed location.

12. The method of claim 1, wherein the indication of identification information associated with the user and the indication of health status information are represented by a first hash included in the personal health status information verification credentials, the first hash being generated based on the indication of identification information associated with the user and the indication of health status information.

13. The method of claim 1, wherein the indication of the first authority comprises a public key associated with the first authority and the indication of the second authority comprises a public key associated with the second authority.

14. The method of claim 13, wherein the indication of the first authority associated with the health status information and the indication of a second authority associated with the system are represented by a second hash included in the personal health status information verification credentials, the second hash being generated based on the indication of the first authority associated with the health status information and the indication of a second authority associated with the system.

15. The method of claim 14, wherein the second hash comprises a hash calculated based on a third hash of the public key associated with the first authority and a fourth hash of the public key associated with the second authority.

16. The method of claim 1, wherein the indication of whether the personal health status information verification credentials are validated by the gatekeeping application comprises an indication provided by an interface of the system.

17. The method of claim 16, wherein the indication of whether the personal health status information verification credentials are validated by the gatekeeping application comprises at least one of a text indication, a visual indication, and an audio indication.

18. The method of claim 1, wherein the method further comprises transmitting the indication of whether the personal health status information verification credentials are validated by the gatekeeping application to the user device.

19. The method of claim 1, wherein each trusted distributed assertion ledger of the one or more trusted distributed assertion ledgers comprises cryptographically linked ledger entries.

20. The method of claim 1, wherein each trusted distributed assertion ledger comprises a blockchain ledger.

21. The method of claim 1, wherein at least one trusted distributed assertion ledger of the one or more trusted distributed assertion ledgers comprises one or more entries derived from at least subset of entries of one or more other trusted ledgers.

22. The method of claim 1, wherein the health status verification query comprises at least one indication of an assertion type associated with the health status verification query.
